(19) United States
(12) Patent Application Publication
(10) Pub. No.: US 2007/0142422 A1
(43) Pub. Date: Jun. 21, 2007

(54) Control of CCI-779 dosage form stability through control of drug substance impurities

(75) Inventors: Joseph Thomas Rubino, Towaco, NJ (US); Pooja Gandhi, Highland Mills, NY (US); Lynn Phelan, Lake Hiawatha, NJ (US)

Correspondence Address: HOWSON AND HOWSON;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 11/642,029

(22) Filed: Dec. 19, 2006

Related U.S. Application Data
(60) Provisional application No. 60/752,189, filed on Dec. 20, 2005.

(51) Int. Cl. A61K 31/4745; A61K 31/355; A61K 31/375

(52) U.S. Cl. 514/291; 514/458; 514/474

(57) ABSTRACT

A method of preparing a rapamycin composition having increased potency is provided. The method involves selecting a rapamycin compound having less than 1.5% oxidative and hydrolytic rapamycin impurities and formulating the selected rapamycin with an antioxidant and optional excipients.

### CROSS-REFERENCE TO RELATED APPLICATION

This application claims the benefit under 35 USC 119(e) of U.S. Provisional Patent Application No. 60/752,189, filed Dec. 20, 2005.

### BACKGROUND OF THE INVENTION

Rapamycin 42-ester with 3-hydroxy-2-(hydroxymethyl)-2-methylpropionic acid (CCI-779) has potential as an antitumor agent. This compound is now known generically under the name temsirolimus (Wyeth). The preparation and use of hydroxyesters of rapamycin, including temsirolimus, are described in U.S. Pat. Nos. 5,362,718 and 6,277,983.

The intravenous dosage form includes a solution of CCI-779, α-tocopherol, citric acid, and ethanol in propylene glycol. Rapamycin and related compounds may be susceptible to oxidative and hydrolytic degradation during synthesis and purification of the drug substance or when formulated as a dosage form. Oxidation generally begins via peroxidation of unsaturated carbons in the 1-7 carbon polyene region of rapamycin and its derivatives, such as CCI-779. The initial peroxidation generally proceeds to form a number of oxide, hydroxide and aldehyde degradation products.

Collectively, these degradation products or impurities are referred to as “group II” or “oxidative and hydrolytic” degradation products or impurities. The presence of these impurities/degradation products can catalyze degradation of the drug and thereby destabilize the drug when present in sufficiently high amounts.

Addition of antioxidants to both the drug substance, during processing, and in the final drug product may inhibit degradation caused by the degradation products or impurities. However, when these degradation/impurity levels reach a critical value, further degradation of drug product is difficult to inhibit by practical means. This is especially a limitation for parenteral products because the levels of antioxidants and other stabilizers used in a formulation is often limited by safety concerns and their levels in new products may be limited by previous human safety experience. Because of the negative influence of oxidative/hydrolytic degradation products on the potency and purity of drug product, it is advantageous to limit their amount in the composition of the final drug product.

U.S. Pat. No. 6,605,613 B2 discusses stabilization of macrolides using various antioxidants. The primary focus of that patent is to stabilize drug during its preparation and final isolation.

Because of the variety of degradation pathways that occur during oxidation and hydrolysis of rapamycin derivatives as well as the ability of rapamycin and related compounds to form various isomers, the isolation and quantitation of oxidative/hydrolytic degradation products/impurities as individual substances is difficult to achieve. For this reason, it is often necessary to quantitate oxidative/hydrolytic degradation products as a group rather than as single, individual compounds.

What is needed in the art are alternate methods for preparing rapamycin compositions that have less degradation impurities.

### SUMMARY OF THE INVENTION

In one aspect, the present invention provides a method of preparing a rapamycin composition having increased potency.

In another aspect, the present invention provides a method for preparing a rapamycin composition having increased potency by formulating a rapamycin compound having not more than 1.5% oxidative and hydrolytic rapamycin impurities with an antioxidant and optional excipients.

Other aspects and advantages of the invention will be readily apparent from the following detailed description of the invention.

### BRIEF DESCRIPTION OF THE DRAWING

FIG. 1 is an LC/UV chromatogram of an exemplary temsirolimus (CCI-779) sample preparation which is corrected for solvent.

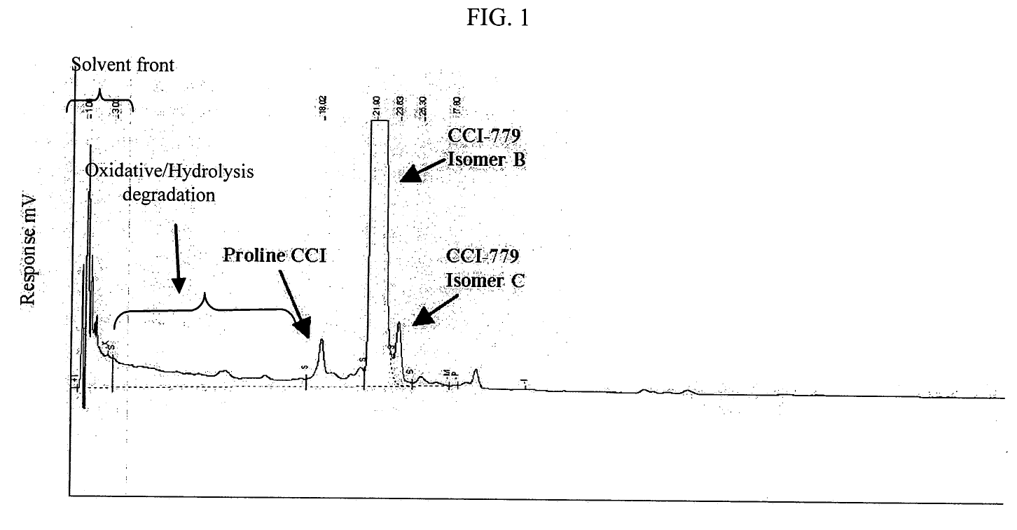

FIG. 2 is an LC/MS chromatogram of an exemplary temsirolimus (CCI-779) sample preparation. More particularly, this HPLC/MS Chromatogram for oxidative/hydrolysis degradants has a time in column (TIC) Range: m/z 1044.7 to 1076.7.

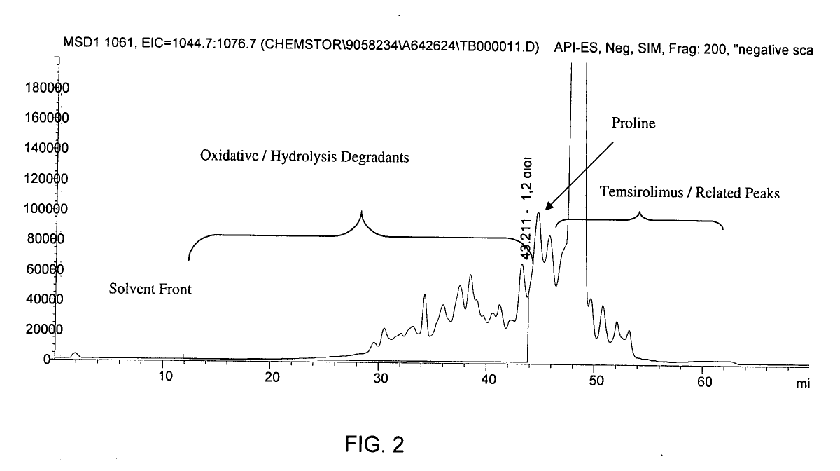

FIG. 3 is a plot of total nonoxidative degradants and oxidative/hydrolysis degradants vs. time for a parenteral drug product of temsirolimus (CCI-779) prepared with drug substance that contained 0.5, 1, or 2% initial oxidative/hydrolysis degradants. In this figure, “OD” refers to the percent (%) oxidative/hydrolysis degradants initially in the drug substance.

### DETAILED DESCRIPTION OF THE INVENTION

The present invention provides methods of preparing rapamycin compositions having increased stability. The term “increased stability” as used herein refers to a rapamycin composition whereby the concentration of the rapamycin compound contained therein decreases to a lesser extent over time and has fewer or lower levels of degradation products as compared to rapamycin compositions in the art. Desirably, the rapamycin compositions of the present invention show minimal degradation after storage at 25° C. or 40° C. compared to compositions where the oxidative/hydrolytic impurities in the starting material are not controlled.

As used herein, the term rapamycin compound defines a class of immunosuppressive compounds that contain the basic rapamycin nucleus as shown below.

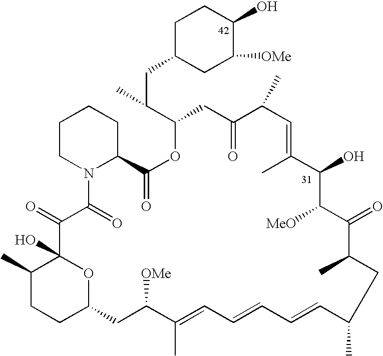

The rapamycin compounds of this invention include compounds that are chemically or biologically modified as derivatives of the rapamycin nucleus, while still retaining immunosuppressive properties. Accordingly, the term rapamycin compound includes rapamycin, and esters, ethers, carbamates, oximes, hydrazones, and hydroxylamines of rapamycin, as well as rapamycins in which functional groups on the rapamycin nucleus have been modified, for example through reduction or oxidation.

The term rapamycin compound also includes 42- and/or 31-esters and ethers of rapamycin as described in the following patents, which are all hereby incorporated by reference: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl esters (U.S. Pat. No. 5,120,842); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). The preparation of these esters and ethers is disclosed in the patents listed above.

Further included within the definition of the term rapamycin compound are 27-esters and ethers of rapamycin, which are discussed in U.S. Pat. No. 5,256,790. Also described are C-27 ketone rapamycins which are reduced to the corresponding alcohol, which is in turn converted to the corresponding ester or ether. The preparation of these esters and ethers is discussed in the patents provided above. Also included are oximes, hydrazones, and hydroxylamines of rapamycin as discussed in U.S. Pat. Nos. 5,373,014; 5,378,836; 5,023,264; and 5,563,145. The preparation of these oximes, hydrazones, and hydroxylamines is discussed in the above-listed patents. The preparation of 42-oxorapamycin is discussed in U.S. Pat. No. 5,023,263.

The term rapamycin compound also refers to any combination of different rapamycins or chemical compounds that contains rapamycin or any derivative thereof.

Specific examples of rapamycin compounds that can be used in the invention include, without limitation, rapamycin, CCI-779, norrapamycin, deoxorapamycin, desmethylrapamycin, desmethoxyrapamycin, or the rapamycins described in US Patent Publication No. 2006-0135549 (claiming priority from US Provisional Application No. 60/637,666) and US Patent Publication No. 2006-013550 A1, (claiming priority from US Provisional Application No. 60/638,004), which are hereby incorporated by reference, or pharmaceutically acceptable salts, prodrugs, or metabolites thereof, among others.

The term “desmethylrapamycin” refers to the class of rapamycin compounds which lack one or more methyl groups. Examples of desmethylrapamycins that can be used according to the present invention include 3-desmethylrapamycin (U.S. Pat. No. 6,358,969), 7-O-desmethyl-rapamycin (U.S. Pat. No. 6,399,626), 17-desmethylrapamycin (U.S. Pat. No. 6,670,168), and 32-O-desmethylrapamycin, among others.

The term “desmethoxyrapamycin” refers to the class of rapamycin compounds which lack one or more methoxy groups and includes, without limitation, 32-desmethoxyrapamycin.

The rapamycin compositions of the invention include the rapamycin compound at an amount sufficient to treat the conditions and diseases identified below. Specifically, the rapamycin compound is present in the rapamycin compositions at about 0.1 to 30 wt %, 0.5 to 25 wt %, 1 to 20 wt %, 5 to 15 wt %, or 7 to 12 wt % (wt/wt). Desirably, the rapamycin compound is present at an amount of 2 to about 500 mg, 5 mg to 250 mg, 10 mg to 100 mg, 15 mg to 50 mg, or about 20 mg to 25 mg.

The rapamycin compound of the invention can be in a micronized or nonmicronized form and can also include tautomeric forms of the rapamycin compound. The present invention also includes derivatives of rapamycin, including, but not limited to, esters, carbamates, sulfates, ethers, oximes, carbonates, and the like.

The rapamycin compound can also encompasses “metabolites” which are unique products formed by processing rapamycin by the cell or patient. Desirably, metabolites are formed in vivo.

It is also desirable that the rapamycin compound in the compositions of the invention degrades less than the rapamycin compound in the compositions in the art. Of course, it is most desirable that the concentration of the rapamycin compound in the compositions of the present invention be maintained. However, it is desirable that the concentration of the rapamycin compound in the compositions of the invention degrades less than about 2% after storage for 3-5 months at 25° C. or 1 month at 40° C., more desirably less than about 1%.

The inventors found that more potent rapamycin compositions are obtained when the rapamycin compound utilized therein contained less than 1.5% (i.e., 0 or 0.01, 0.01 to 1.5%) oxidative and hydrolytic impurities in the starting material. In fact, by utilizing rapamycin compounds containing 0.5 to 1% or less oxidative and hydrolytic impurities, degradation of the rapamycin was significantly reduced or eliminated. More desirably, the rapamycin compound contains less than about 1%, less than about 0.5%, less than about 0.4%, less than about 0.3%, less than about 0.2%, or about 0.1% oxidative impurities. Most desirably, the rapamycin contains less than about 0.5% oxidative impurities.

The term “oxidative and hydrolytic impurities of rapamycin”, or variations thereof as used herein, refers to chemical compounds that form in rapamycin compositions. These impurities can include a group of oxygen addition compounds involving the C₁₋₆ region of rapamycin or its analogs identified below. These impurities can therefore include aldehydes, epoxides, hydroxides, and combinations thereof of rapamycin or rapamycin derivatives. These impurities can also include ring-opened forms of rapamycin or rapamycin analogs that contain the oxygen addition modifications described above for the C₁₋₆ region.

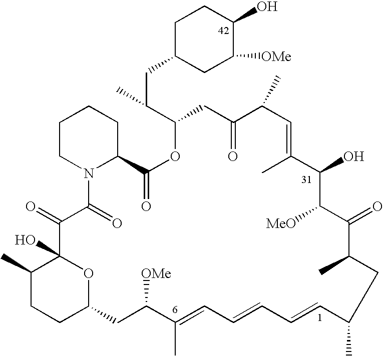

The presence of these oxidative and hydrolytic impurities is typically measured using high performance liquid chromatography (HPLC) with ultraviolet (UV) or mass spectrometric (MS) detection. Specifically, the oxidative and degradation impurities can be quantitated using either HPLC/UV or HPLC/MS. More specifically, the oxidative and hydrolytic impurities may be quantitated as a mixture of co-eluting materials over a specified range of retention times (HPLC/UV). For example, in one embodiment, the retention time of the CCI-779 Isomer B peak should be between 18 and 24 minutes using a suitable chromatograph column (e.g., a reverse phase column). Alternatively quantitation by analyzing the extent of one oxygen, two oxygen, 3 oxygen, one oxygen plus water, and water incorporation, based on the m/z of the addition product, is employed.

The method of the invention thereby includes preparing a rapamycin composition by selecting a rapamycin compound as noted above for use therein. Desirably, the rapamycin compound has less than 1.5% oxidative and hydrolytic rapamycin impurities. After selection of the desired rapamycin compound, it is formulated with one or more of an antioxidant.

Antioxidants that can be used in the rapamycin compositions of the present invention include, but are not limited to, citric acid, alpha tocopherol, BHA, BHT (2,6-di-tert-butyl-4-methylphenol), monothioglycerol, Vitamin C, and propyl gallate. In one embodiment, Vitamin C is ascorbic acid. However, one of skill in the art may substitute a pharmaceutically acceptable salt thereof for the ascorbic acid. Desirably, the antioxidant is d,l-α-tocopherol. In one embodiment, the antioxidant may be used in concentrations ranging from 0.0005 wt % to 3 wt %, and desirably from 0.001 wt % to 3 wt %.

The rapamycin compositions of the invention may also contain suitable excipients including, without limitation, water soluble polymers, pH modifying agents, chelating agents, surfactants, fillers, binders, disintegrants, and the like. Any given rapamycin composition useful in the invention may contain multiple ingredients of each class of component. For example, some compositions may contain one or more antioxidant.

pH modifying agents include, but are not limited to, citric acid, sodium citrate, acetic acid, lactic acid, dilute HCl, and other mild acids or bases capable of buffering a solution containing the rapamycin compound to a pH in the range of about 4 to about 6.

Chelating agents, and other materials capable of binding metal ions, can be included in the rapamycin compositions of the invention. Desirably, the chelating agent enhances the stability of the rapamycin compound. In certain embodiments, the antioxidant component of the formulation of the invention can exhibit chelating activity. Examples of chelating agents include, without limitation, citric acid and ascorbic acid (which may function as both a classic antioxidant and a chelating agent in the present formulations). Other chelating agents include such materials as are capable of binding metal ions in solution, such as ethylene diamine tetra acetic acid (EDTA), its salts, or amino acids such as glycine, which are capable of enhancing the stability of the rapamycin compound. Typically, chelating agents are used in the lower end of the range of concentrations for the antioxidant component provided herein. In one example, citric acid is utilized at a concentration of less than 0.01% w/v. Additionally, such chelating agents may be used in combination with other antioxidants as part of the antioxidant component of the invention. For example, an acceptable formulation may contain both citric acid and d,l-α-tocopherol. Optimal concentrations for the selected antioxidant(s) can be readily determined by one of skill in the art, based upon the information provided herein.

Surfactants may include polysorbate 80, polyoxyethylene fatty acid esters, sodium lauryl sulfate, sodium dodecyl sulfate, salts of bile acids (taurocholate, glycocholate, cholate, deoxycholate, etc.) that may be combined with lecithin, Vitamin E TPGS, and/or poloxamers. The surfactant can be present in the rapamycin compositions at 0.5 to 10 wt %, 1 to 8 wt %, or 3 to 5 wt % (wt/wt), or can be present in amounts from the lower or higher end of these ranges up to about 50 wt %.

Binders, fillers, and disintegrants can include sucrose, lactose, microcrystalline cellulose, croscarmellose sodium, magnesium stearate, gum acacia, cholesterol, tragacanth, stearic acid, gelatin, casein, lecithin (phosphatides), carboxymethylcellulose calcium, carboxymethylcellulose sodium, methylcellulose, hydroxyethylcellulose, hydroxypropylcellulose, hydroxypropylmethycellulose phthalate, noncrystalline cellulose, cetostearyl alcohol, cetyl alcohol, cetyl esters wax, dextrates, dextrin, lactose, dextrose, glyceryl monooleate, glyceryl monostearate, glyceryl palmitostearate, polyoxyethylene alkyl ethers, polyethylene glycols, polyoxyethylene castor oil derivatives, polyoxyethylene stearates, and polyvinyl alcohol.

Typical water soluble polymers include, but are not limited to, polyvinylpyrrolidone (PVP), hydroxypropylmethylcellulose (HPMC), polyethylene glycol (PEG), and cyclodextrin or mixtures thereof. Desirably, the water-soluble polymer is PVP and has a molecular weight of between 2.5 and 60 kilodaltons.

The rapamycin compositions described herein can be formulated in any form suitable for the desired route of delivery using a pharmaceutically effective amount of the rapamycin compound. For example, the compositions of the invention can be delivered by a route such as oral, dermal, transdermal, intrabronchial, intranasal, intravenous, intramuscular, subcutaneous, parenteral, intraperitoneal, intranasal, vaginal, rectal, sublingual, intracranial, epidural, intratracheal, or by sustained release.

Suitable oral formulations for the rapamycin compositions can be prepared as described for CCI-779, as described in International Patent Publication No. WO 2004/026280 and US Patent Application Publication No. US 2004-0077677 A1, which are hereby incorporated by reference. In one embodiment, the composition contains 0.1 to 30 wt %, 0.5 to 25 wt %, 1 to 20 wt %, 5 to 15 wt %, or 7 to 12 wt % (wt/wt) of a rapamycin compound and 0.001 wt % to 1 wt %, 0.01 wt % to 1 wt %, or 0.1 wt % to 0.5 wt % (wt/wt) of an antioxidant. The compositions can optionally contain 0.5 to 50 wt %, 1 to 40 wt %, 5 to 35 wt %, 10 to 25 wt %, or 15 to 20 wt % (wt/wt) of a water soluble polymer and 0.5 to 10 wt %, 1 to 8 wt %, or 3 to 5 wt % (wt/wt) of a surfactant. However, other embodiments may contain more, or less, of these components.

Oral formulations may include any conventionally used oral forms, including tablets, capsules, buccal forms, troches, lozenges and oral liquids, suspensions or solutions. Capsules may contain mixtures of the rapamycin compound with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Surface modifying agents can include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the rapamycin. The oral formulation may also include water or a fruit juice, containing appropriate solubilizers or emulsifiers as needed.

In some cases it may be desirable to administer the rapamycin composition directly to the airways in the form of an aerosol.

The rapamycin compositions may also be administered parenterally or intraperitoneally. Solutions or suspensions of the rapamycin compound as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Particularly suitable injectable formulations containing the rapamycin compound can be prepared in a manner similar to those described in International Patent Publication No. WO 2004/011000 and US Patent Application Publication No. US 2004-0167152 A1, which are hereby incorporated by reference. In this embodiment, the injectable formulation useful in the invention provides a rapamycin composition cosolvent concentrate containing a parenterally acceptable solvent and an antioxidant as described above and a parenteral formulation containing a rapamycin compound, a parenterally acceptable cosolvent, an antioxidant, a diluent solvent, and a surfactant. For example, a parenterally acceptable solvent can include a non-alcoholic solvent, an alcoholic solvent, or mixtures thereof. Examples of suitable non-alcoholic solvents include, without limitation, dimethylacetamide, dimethylsulfoxide, or mixtures thereof. Examples of alcoholic solvent include, without limitation, one or more alcohols as the alcoholic solvent component of the formulation. Examples of solvents useful in the formulations invention include, without limitation, ethanol, propylene glycol, polyethylene glycol 300, polyethylene glycol 400, polyethylene glycol 600, polyethylene glycol 1000, or mixtures thereof. Further, ethanol and propylene glycol can be combined to produce a less flammable product. These latter two cosolvents are particularly desirable because degradation via oxidation and lactone cleavage occurs to a lower extent for these cosolvents. Larger amounts of ethanol in the mixture generally result in better chemical stability. A concentration of 30 to 100% v/v of ethanol in the mixture is desirable.

The stability of the rapamycin compound in the parenterally acceptable alcoholic cosolvent can be enhanced by addition of an antioxidant to the formulation. Generally, the parenteral formulation useful in the invention will contain an antioxidant component(s) in a concentration of 0.001% to 3% w/v or 0.01% to 0.1% w/v, of the cosolvent concentrate, although lower or higher concentrations may be desired. Of the antioxidants, d,l-α-tocopherol is particularly desirable and is used at a concentration of 0.01 to 0.1% w/v with a concentration of 0.075% w/v of the cosolvent concentrate being most-desirable.

Dosage regimens are expected to vary according to the route of administration. For example, dosages for oral administration are often up to five to tenfold greater than for intravenous (i.v.) administration. In one embodiment, a dosage of the rapamycin compound may be about 2 to about 500 mg/day, 5 mg/day to 75 mg/day, 10 mg/day to 50 mg/day, 15 mg/day to 35 mg/day, or about 20 mg/day to 25 mg/day for an adult. However, this dosage can be adjusted upwardly or downwardly by one of skill in the art, depending upon the indication being treated, the size of the patient, and other factors which are known those of skill in the art.

In certain embodiments of the parenteral formulations useful in the invention, precipitation of the rapamycin compound upon dilution with aqueous infusion solutions or blood is prevented through the use of a surfactant contained in the diluent solution. The most important component of the diluent is a parenterally acceptable surfactant. One particularly desirable surfactant is polysorbate 20 or polysorbate 80. However, one of skill in the art may readily select other suitable surfactants from among salts of bile acids (taurocholate, glycocholate, cholate, deoxycholate, etc.) which are optionally combined with lecithin. Alternatively, ethoxylated vegetable oils, such as a pegylated castor oil (e.g., such as PEG-35 castor oil which is sold, e.g., under the name Cremophor EL, BASF), vitamin E tocopherol propylene glycol succinate (Vitamin E TGPS), and polyoxyethylene-polyoxypropylene block copolymers can be used in the diluent as a surfactant, as well as other members of the polysorbate family such as polysorbate 20 or 60. Other components of the diluent may include water, ethanol, polyethylene glycol 300, polyethylene 400, polyethylene 600, polyethylene 1000, or blends containing one or more of these polyethylene glycols, propylene glycol and other parenterally acceptable cosolvents or agents to adjust solution osmolarity such as sodium chloride, lactose, mannitol or other parenterally acceptable sugars, polyols and electrolytes. It is expected that the surfactant will include at least 5% w/v, at least 10% w/v, or at least 5% w/v of the diluent solution. Desirably, the surfactant will include 2 to 100% w/v, 5 to 80% w/v, 10 to 75% w/v, or 15 to 60 % w/v of the diluent solution.

A parenteral formulation useful in the invention can be prepared as a single solution, or desirably can be prepared as a cosolvent concentrate containing the rapamycin compound, an alcoholic solvent, and an antioxidant, which is subsequently combined with a diluent that contains a diluent solvent and suitable surfactant. Prior to use, the cosolvent concentrate is mixed with a diluent comprising a diluent solvent, and a surfactant. When rapamycin compound is prepared as a cosolvent concentrate according to this invention, the concentrate can contain concentrations of the rapamycin compound from 0.05 mg/mL, from 2.5 mg/mL, from 5 mg/mL, from 10 mg/mL or from 25 mg/mL up to approximately 50 mg/mL. The concentrate can be mixed with the diluent up to approximately 1 part concentrate to 1 part diluent, to give parenteral formulations having concentrations of the rapamycin compound from 1 mg/mL, from 5 mg/mL, from 10 mg/mL, from 20 mg/mL, up to approximately 25 mg/mL. For example the concentration of the rapamycin compound in the parenteral formulation may be from about 2.5 to 10 mg/mL. This invention also covers the use of formulations having lesser concentrations of the rapamycin compound in the cosolvent concentrate, and formulations in which one part of the concentrate is mixed with greater than 1 part of the diluent, e.g., concentrate: diluent in a ratio of about 1: 1.5, 1:2, 1:3, 1:4 ,1:5, or 1:9 v/v and so on, to rapamycin compound parenteral formulations having a rapamycin compound concentration down to the lowest levels of detection. Typically the antioxidant may include from about 0.0005 to 0.5% w/v of the formulation. The surfactant may for example include from about 0.5% to about 10% w/v of the formulation. The alcoholic solvent may for example include from about 10% to about 90% w/v of the formulation.

The parenteral formulations useful in this invention can be used to produce a dosage form that is suitable for administration by either direct injection or by addition to sterile infusion fluids for intravenous infusion.

Transdermal administrations include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal). Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

The rapamycin compound of the invention may be formulated for any suitable delivery route and vehicle and assembled in the form of a kit of parts.

Thus, the rapamycin compositions of the invention can be useful as an antineoplastic agent, and therefore in the treatment of solid tumors, including sarcomas and carcinomas; and more particularly against astrocytomas, prostate cancer, breast cancer, colon cancer, small cell lung cancer, and ovarian cancer; and adult T-cell leukemia/lymphoma. The rapamycin compound-containing compositions are also useful in the treatment or inhibition of transplantation rejection such as kidney, heart, liver, lung, bone marrow, pancreas (islet cells), cornea, small bowel, and skin allografts, and heart valve xenografts; in the treatment or inhibition of graft vs. host disease; in the treatment or inhibition of autoimmune diseases such as lupus, rheumatoid arthritis, diabetes mellitus, myasthenia gravis, and multiple sclerosis; and diseases of inflammation such as psoriasis, dermatitis, eczema, seborrhea, inflammatory bowel disease, pulmonary inflammation (including asthma, chronic obstructive pulmonary disease, emphysema, acute respiratory distress syndrome, bronchitis, and the like) and ocular uveitis; adult T-cell leukemia/lymphoma; fungal infections; hyperproliferative vascular diseases such as restenosis; graft vascular atherosclerosis; and cardiovascular disease, cerebral vascular disease, and peripheral vascular disease, such as coronary artery disease, cereberovascular disease, arteriosclerosis, atherosclerosis, nonatheromatous arteriosclerosis, or vascular wall damage from cellular events leading toward immune mediated vascular damage, and inhibiting stroke or multiinfarct dementia.

The following examples are illustrative only and are not intended to be a limitation on the present invention.

### EXAMPLES

### Example 1

### General Procedures for Evaluation of Oxidative Impurities Present in CCI-779 Samples

The oxidative and hydrolytic impurities in a CCI-779 sample may be quantitated as a mixture of co-eluting materials over a specified range of retention times. The method used is a reverse phase gradient HPLC/UV procedure. The chromatographic conditions are outlined below.

TABLE 1
Chromatographic Conditions (HPLV/UV) for Determination of
Oxidative/Hydrolysis Degradation Products

| Method Parameter | Requirement | |
|---|---|---|
| Column Specification: | Ultracarb ™ ODS(30), 150 × 4.6 mm, 5 μm size | |
| | particles | |
| Mobile Phase: | Mobile Phase A: 50% acetonitrile: 50% water | |
| | Mobile Phase B: 80% acetonitrile: 20% water | |
| Gradient: | Time | % Mobile Phase B |
| | 0 | 0 |
| | 40 | 100 |
| | 60 | 100 |
| | 60.1 | 0 |
| | 75 | 0 |

| | |
|---|---|
| Column Temperature: | 45° C. |
| Flow Rate: | 1.5 mL/minute |
| Detection: | UV, 225 nm |
| Injection Volume: | 50 μL |
| Sample solvent | 300 mL water: 700 mL acetonitrile: 0.5 mL acetic acid |
| Sample Preparation: | 2.0 mL of sample is transferred into a 25 mL |
| | volumetric flask and diluted to volume with sample |
| | solvent |
| Calibration: | The amount of oxidation/hydrolysis degradation |
| | products is determined by comparing the appropriate |
| | peak areas in the sample preparation chromatograph to |
| | the total area of CCI-779 and its related compounds. |
| | The area between RRT^{a} 0.13 to 0.81 is a measure of the |
| | oxidation/hydrolysis degradation products. The |
| | reporting limit was 0.05% |
| | a. RRT = Relative retention time. |
| | Retention time of isomer B used as reference point |
| System Suitability Preparations | 20 μg/mL of Temsirolimus reference standard in |
| | sample solvent. (solution A) |
| | 1 μg/mL of Temsirolimus reference standard in sample |
| | solvent. (solution B) |
| | 2 mg/mL of a Temsirolimus control batch in sample |
| | solvent. |
| System Suitability | |
| Sample solvent blank | No interfering peaks between 4-19 minutes |
| Signal to Noise (Solution B) | S/N >10 |
| Retention time CCI-779 Isomer B | 18 to 24 minutes |
| (Solution A) | |
| Theoretical Plates CCI-779 Isomer B | ≧2000 |
| (Solution A) | |
| Tailing Factor CCI-779 Isomer B | ≦2.0 |
| (Solution A) | |
| % CV (blank subtracted control | ≦10% |
| samples) | |
| | ≧100 |
| % RSD (6 standard injections): | ≦6.0% |

Table 2

With reference to Table 1 above, the following table provides further details relating to the gradients used with mobile phase A (MP-A) and mobile phase B (MP-B). During “linear change”, the gradient is changed at a uniform rate. During “isocratic hold”, the solvent phase remains constant. During “step change”, an incremental change in the solvent is introduced.

| Time (min) | % MP-A | % MP-B | Comments |
|---|---|---|---|
| 0 | 100 | 0 | Initial conditions |
| 40 | 0 | 100 | Linear change |
| 60 | 0 | 100 | Isocratic hold |
| 60.1 | 100 | 0 | Step change |
| 75 | 100 | 0 | Equilibrate for next injection |

As an alternative to HPLC/UV, the CCI-779 in the sample was quantitated by analyzing the extent of one oxygen, two oxygen, 3 oxygen, one oxygen plus water, and water incorporation, based on the m/z of the addition product.

TABLE 3
Chromatographic Mass Spectrometric (HPLC/MS) Conditions for
Determination of Oxidative/Hydrolysis Degradation Products

| Method Parameter | Requirement | | |
|---|---|---|---|
| Column Specification | C18, 3 μm or 5 μm,150 × 2.0 mm held at 45° C. | | |
| Mobile Phase | Mobile Phase A: Mix 95 volumes of water with | | |
| | 5 volumes of acetonitrile and 0.1 volume of | | |
| | formic acid. | | |
| | Mobile Phase B: Mix 5 volumes of water with | | |
| | 95 volumes of acetonitrile and 0.1 volume of formic acid. | | |
| Gradient | Gradient: | Time (min) | % B |
| | | 0 | 30 |
| | | 6 | 30 |
| | | 46 | 76 |
| | | 47 | 100 |
| | | 57 | 100 |

| | |
|---|---|
| | Re-equilibrate at initial conditions for 13 minutes |
| Flow Rate | 0.2 mL/min |
| Detection | Mass spectrometric |
| Ionization Mode | Negative Electrospray |
| Single Ion Monitoring | m/z 1014.7, m/z 1044.7, m/z 1046.71046, m/z 1060.7, m/z |
| | 1062.7 and m/z 1076.7 |
| Sample Solvent | Acetonitrile (API) |
| | 30:70:0.5 (water:acetonitrile:acetic acid V:V:V) for Drug |
| | product |
| Standard | 60 μg/mL, 50 μg/mL, 40 μg/mL, 30 μg/mL, 10 μg/mL, 4 μg/mL |
| | and 1 μg/mL of 1R,2R-Dihydroxyl Temsirolimus |
| Calibration | Calculate the quadratic regression line relating the peak areas (y) |
| | of the 1R,2R-Dihydroxyl Temsirolimus peak in the standard |
| | preparation chromatograms to the corresponding concentration |
| | value of the reference standard (x). Connect the low standard |
| | point to the origin. |
| Control Preparation | Temsirolimus preparation at a concentration of about 2 mg/Ml |
| System Suitability | |
| Check Tune | Must pass for the range of m/z 800-1100 |
| Retention time 1R,2R, | 30 to 38 minutes |
| Dihydroxyl temsirolimus | |
| (30 μg/mL standard) | |
| Theoretical Plates 1R,2R, | 10,000 |
| Dihydroxyl temsirolimus | |
| (30 μg/mL standard) | |
| Tailing Factor 1R,2R, | ≦2.0 |
| Dihydroxyl temsirolimus | |
| (30 μg/mL standard) | |
| Signal to noise | ≧10 |
| (1 μg/mL standard) | |
| R² for the quadratic | ≧0.990 |
| regression | |

### Example 2

### Varying Levels of Impurities

In this example, three rapamycin compositions, each of which contains CCI-779 2.5%, d,l-alpha-tocopherol 0.075%, anhydrous citric acid 0.0025%, dehydrated alcohol 39.5%, and propylene glycol q.s., with varying levels of oxidative impurities, were monitored over a period of about 3 to 5 months to determine their stabilities at various temperatures and humidities. These batches contained about 0.5%, about 1% and about 2% of oxidative/hydrolysis impurities, respectively.

Aliquots of the formula were subdivided into glass vials, stoppered, sealed and stored at 5° C., 25° C./60% relative humidity (RH), or 40° C./75% RH. Samples were monitored for (i) appearance and description, (ii) moisture, (iii) strength, total related compounds (nonoxidative), (iv) oxidative/hydrolysis impurities, and (v) α-tocopherol content. The data illustrates that for samples initially containing about 0.5% impurities, there was a slight increase in total (nonoxidative) degradation and oxidative/hydrolytic degradation after 1 month at 40° C. After 3 and 5 months at 25° C. the formulation was stable, i.e., the potency remained the same as did total degradation.

For the sample initially containing about 1% impurities, there was an increase in oxidative/hydrolytic degradation products to about 1.94% after three months. This trend continued out to 5 months at 25° C., with increases in total nonoxidative and oxidative/hydrolytic degradation products to 1.65 and 2.3% respectively.

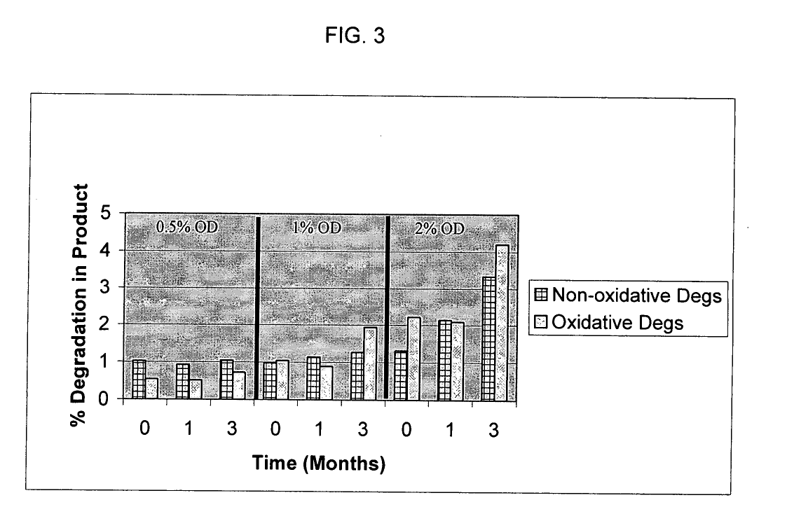

For the sample initially containing about 2% impurities, the total nonoxidative degradation and oxidative/hydrolytic degradation increased after 1 month 40° C./75% RH to 8 and 4.3% respectively. After three months, both total nonoxidative degradation and oxidative/hydrolytic degradation products increased for the samples at 25° C./60% RH to 3.3 and 4.2% respectively. FIG. 3 illustrates the effect of initial oxidative/hydrolysis degradant levels contributed by the input drug raw material on the stability of CCI-779 after 1 and 3 month storage.

In summary, it was found that higher initial concentrations of the oxidative/hydrolytic impurities adversely affected the stability of the CCI-779 samples. In fact, the greatest stability of samples containing CCI-779 occurred when the initial concentration of the oxidative impurities in the rapamycin composition was 0.5% or less. Therefore, reducing the initial oxidative impurities significantly enhances the shelf-life of the formulated CCI-779 product.

### Example 3

### Varying α-Tocopherol Concentration

To further investigate the effect of the oxidative impurities in rapamycin compositions, studies were conducted by varying concentrations of α-tocopherol in the rapamycin compositions.

Samples of CCI-779 containing 0.2%, 0.5% and 1% d,l-α-tocopherol (Eisai) were placed in 2 mL flint glass vials and stoppered with 13 mm West Teflon Faced 4432/50 stoppers. The effect of increased a-tocopherol concentrations on the rapamycin compositions was monitored over 1 month at 40° C. Samples were stored upright at about 5° C. or about 40° C.

After 1 month at 40° C., the data illustrate that in all of the samples, the α-tocopherol concentration dropped significantly. However, for the samples containing 0.2% and 0.5% of α-tocopherol, the concentration of the oxidative impurities remained essentially unchanged, i.e., the concentration of oxidative impurities did not increase. However, there was an overall loss of potency of the samples due to the formation of other degradation products.

For samples containing 1% α-tocopherol, the presence of the oxidative impurities drastically increased to 8.42%.

In summary, increasing the concentration of α-tocopherol in the samples to 0.2 and 0.5% slowed the growth of oxidative impurities, but did not inhibit degradation of the CCI-779 via other mechanisms when the oxidative impurity levels were 3% or more. As indicated in the previous example, inhibition of the non-oxidative impurities was controlled by limiting the initial amount of oxidative/hydrolysis impurities in the drug substance.

All documents listed in this specification are incorporated herein by reference. While the invention has been described with reference to particular embodiments, it will be appreciated that modifications can be made without departing from the spirit of the invention. Such modifications are intended to fall within the scope of the appended claims.

What is claimed is:

1. A method of preparing a rapamycin composition having increased potency, said method comprising the steps of:
selecting a rapamycin compound having less than 1.5% oxidative and hydrolytic rapamycin impurities; and
formulating the selected rapamycin with an antioxidant and optional excipients.

2. The method according to claim 1, wherein the selecting step comprises screening the rapamycin in a high performance liquid chromatography assay.

3. The method according to claim 1, wherein the antioxidant is selected from the group consisting of a tocopherol, vitamin C, 2,6-di-tert-butyl-4-methylphenol, and mixtures thereof.

4. The method according to claim 3, wherein the antioxidant is α-tocopherol.

5. The method according to claim 1, wherein the selected rapamycin has less than 0.5% oxidative impurities.

6. The method according to claim 1, wherein the selected rapamycin is formulated for parenteral delivery.

7. The method according to claim 1, wherein the selected rapamycin is formulated as a liquid concentrate.

8. The method according to claim 7, wherein the selected rapamycin is formulated with d,l-α-tocopherol, anhydrous citric acid, dehydrated alcohol, and propylene glycol.

9. The method according to claim 1, wherein the selected rapamycin is formulated for oral delivery.

10. A method of preparing a rapamycin composition having increased potency, said method comprising the steps of:
selecting a rapamycin compound having less than 1.5% oxidative and hydrolytic rapamycin impurities;
formulating the selected rapamycin with at least two antioxidants and optional excipients.

11. The method according to claim 10, wherein at least one of the antioxidants is vitamin C or 2,6-di-tert-butyl-4-methylphenol.

12. The method according to claim 10, wherein said at least two antioxidants are vitamin C and 2,6-di-tert-butyl-4-methylphenol.

13. The method according to claim 1, wherein said rapamycin is selected from the group consisting of rapamycin and CCI-779.
